(19) United States
(12) Patent Application Publication
(10) Pub. No.: US 2015/0272500 A1
(43) Pub. Date: Oct. 1, 2015

(54) COMFORTABLE AND PERSONALIZED MONITORING DEVICE, SYSTEM, AND METHOD FOR DETECTING PHYSIOLOGICAL HEALTH RISKS

(71) Applicant: NIGHT-SENSE, LTD, Givataym (IL)

(72) Inventors: Gadi Kan-tor, Givataym (IL); Shy Hefetz, Ramat Gan (IL); Yoav Ken-Tor, Rehovot (IL)

(73) Assignee: NIGHT-SENSE, LTD, Petah Tikvah (IL)

(21) Appl. No.: 14/435,933

(22) PCT Filed: Oct. 14, 2013
(86) PCT No.: PCT/IB2013/059350

§ 371 Date: Apr. 15, 2015

Related U.S. Application Data
(60) Provisional application No. 61/714,276, filed on Oct. 16, 2012.

(51) Int. Cl. A61B 5/00; A61B 5/11; A61B 5/0488; A61B 5/0205

(52) U.S. Cl. CPC A61B 5/681; A61B 5/02055; A61B 5/746; A61B 5/4266; A61B 5/1118; A61B 5/0488; A61B 5/7264; A61B 5/0022; A61B 5/7282; A61B 5/02405

(57) ABSTRACT

The physiological monitoring device, system, and method disclosed herein is convenient and comfortable to use in the detection of physiological health risks. Embodiments may be coupled to a user's body at one instead of at multiple locations. Also, embodiments may be calibrated to the individual users to minimize the occurrence of false alarms while remaining sensitive enough to detect true physiological risk events. Physiological parameters that are monitored may include of heart rate, heart rate variability, respiration, perspiration, skin temperature, difference between skin and ambient temperatures, motoric activity, and electrical activity in muscles.

### RELATED APPLICATION

This application claims priority under 35 U.S.C. §119(e) and other applicable provisions of law to U.S. Provisional Application No. 61/714,276, filed Oct. 16, 2012, which is hereby incorporated by reference in its entirety.

### BACKGROUND

Many instruments developed over the years to monitor physiological health risks, such as hypoglycemia, asthmatic seizure, epileptic seizure, cardiac arrhythmia, and pulmonary edema as non-limiting examples, require calibration appropriate to the individual person under observation.

Setting the appropriate calibration is even more important for a monitoring instrument to operate properly while the observed person sleeps. Certain dangerous physiological conditions can be sensed even without a monitoring instrument by the person who is awake, but while the person sleeps the condition may persist too long, if no alarm triggers when appropriate. Also, a person pestered by too many false alarms may decide not to use the health monitoring instrument and essentially increase the risk of failing to receive appropriate treatment for a dangerous physiological event. Encouraging user compliance with health monitoring requirements is already challenged for systems employing instruments that are bulky, uncomfortable, or cumbersome to wear or otherwise difficult to couple to the user's body, so repeated false alarms unfortunately motivate users to become less diligent in using their monitoring instruments.

Accordingly, a need exists for a physiological monitoring device, system, or method that is convenient/comfortable to use and is easy to calibrate to the individual user such that the occurrence of false alarms is minimized while true physiological risk events are detected.

### SUMMARY

The present inventors have responded to the need for a physiological monitoring device, system, or method that is convenient/comfortable to use. The technology may be employed for coupling to a user in one place on the user's body instead of coupling at multiple locations, and embodiments may be as comfortable to wear as a wristwatch. Also, as detailed below, the inventors also developed a way to calibrate embodiments to the individual users to minimize the occurrences of false alarms while detecting true physiological risk events.

The invention may be embodied as a non-invasive device for monitoring physiological conditions, the device having a platform, one or more sensors, and a data path. The platform is configured for coupling to a limb of a monitored person. The one or more sensors are mounted on the platform, and the sensors operative to generate data based on physiological parameters of the monitored person. Sensor data flow through the data path to processing circuitry that determines whether to activate an alarm that the monitored person's health is at risk. The determination of the processing circuitry is based on the sensor data and on additional data including data based on physiological parameters of people other than the monitored person, and the additional data is updated repeatedly.

The invention may alternatively be embodied as a non-invasive device for monitoring physiological conditions, the device a platform and a data path. The platform is configured for coupling to a limb of a monitored person, and the platform is also configured for mounting one or more sensors thereon, the sensors being operative to generate data based on physiological parameters of the monitored person. Sensor data flow through the data path to processing circuitry that determines whether to activate an alarm that the monitored person's health is at risk. The determination of the processing circuitry is based on the sensor data and on additional data including data based on physiological parameters of people other than the monitored person, the additional data being updated repeatedly.

The invention may also be embodied as a non-invasive method of monitoring physiological conditions. The method includes: receiving data from one or more sensors mounted to a platform that is coupled to a limb of a monitored person, the sensors generating data based on physiological parameters of the monitored person; and processing the data to determine whether to activate an alarm that the monitored person's health is at risk, the determination being based on the sensor data and on additional data including data based on physiological parameters of people other than the monitored person, the additional data being updated repeatedly.

The invention may further be embodied as a method of assisting the monitoring of physiological conditions. The method includes: receiving data based on physiological parameters of a monitored person, the data being generated by one or more sensors mounted to a platform that is coupled to a limb of the monitored person; receiving data based on physiological parameters of people other than the monitored person; processing the data based on the physiological parameters of the monitored person and the data based on physiological parameters of people other than the monitored person to produce output data; and sending the output data to processing circuitry that determines whether to activate an alarm that the monitored person's health is at risk.

Embodiments of the present invention are described in detail below with reference to the accompanying drawings, which are briefly described as follows:

### BRIEF DESCRIPTION OF THE DRAWINGS

The invention is described below in the appended claims, which are read in view of the accompanying description including the following drawings, wherein:

FIG. 1 provides a conceptual diagram of a monitoring system in accordance with embodiments of the invention;

FIGS. 2A-2C provide illustrations of various views of the invention embodied as a non-invasive monitoring device;

FIG. 3 illustrates a relationship between elements of particular embodiments of the invention;

FIG. 4 provides an illustration of the invention embodied as a non-invasive monitoring system;

FIG. 5 provides a flow chart representing a non-invasive method of monitoring physiological conditions in accordance with embodiments of the invention;

FIG. 6 provides a flow chart representing a method of assisting the monitoring of physiological conditions in accordance with embodiments of the invention;

FIG. 7 illustrates a system having a server computer that operates according to the method represented in FIG. 6; and

FIGS. 8.1-8.12 are reproductions of FIGS. 1-12 of the U.S. provisional patent application upon which the present application claims priority.

### DETAILED DESCRIPTION

The invention summarized above and defined by the claims below will be better understood by referring to the present detailed description of embodiments of the invention. This description is not intended to limit the scope of claims but instead to provide examples of the invention.

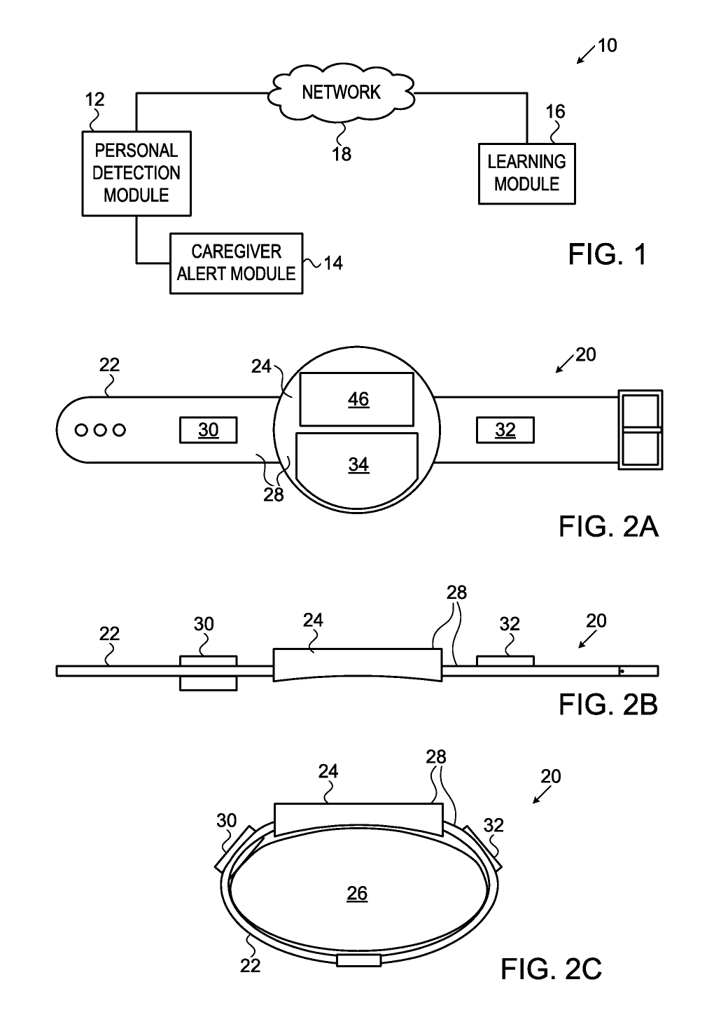

Embodiments of the invention may be understood conceptually as a monitoring system 10 of multiple modules as diagrammed in FIG. 1. A personal detection module 12 positions physiological sensors against or near a user, referenced often hereinafter as the “monitored person,” and determines when and if the monitored person requires medical attention. The personal detection module 12 may activate an alarm to notify the monitored person of the present health condition, thereby causing him/her to react appropriately, or the personal detection module 12 may instead activate an alarm to notify a caregiver that a need exists to treat the monitored person immediately. In the latter scenario, the personal detection module 12 sends a signal to a caregiver alert module 14, which activates the alarm for the caregiver. The caregiver alert module 14 may reside in a nursing station of a hospital or in a caretaker's presence in the home of the user, as non-limiting examples.

Embodiments of the invention adapt to individual users' personal attributes to reduce the occurrences of false alarms while remaining sensitive enough to activate an alarm when necessary. A particular combination of physiological parameters for one person may indicate that medical care is necessary, while for another person the same combination of physiological parameters would not justify activation of an alarm. Accordingly, to reduce the instances of false alarm while nonetheless maintaining a system that is sensitive enough to detect genuine emergencies, the monitoring system 10 implements a learning module 16, which processes in an elaborate fashion data from multiple sources to “calibrate” the monitoring system 10 to the individual user. More details of the processing are provided below.

The diagram of FIG. 1 shows that in the monitoring system 10 the personal detection module 12 and the learning module 16 communicate with each other through a network 18. In a hospital or clinic setting, the network 18 may be a local area network (LAN) enabling a single learning module 16 to process data for multiple personal detection modules, each associated with a different patient. Alternately, the learning module 16 may reside on a server and the network 18 may be the Internet, thereby enabling the learning module 16 to communicate with personal detection modules essentially wherever Internet access is available. In still other embodiments, the network 18 is omitted, and the personal detection module 12 and the learning module 16 communicate with each other directly.

Regarding a personal detection module, FIGS. 2A-2B illustrate a top view and a side view, respectively, of the invention embodied as a non-invasive monitoring device 20 that monitors physiological conditions. As the two drawings along with FIG. 2C show, the monitoring device 20 resembles a wristwatch in that a band 22 (or strap) positions a chassis 24 mounted to the band 22 against a user's wrist 26. The band 22 may be formed of ordinary materials, such as metal, leather, cloth, rubber, or plastic. The band 22 may have a “C” shape as opposed to an “O” shape to clamp to the limb. The chassis 24 and the band 22 collectively form a platform 28 for sensors and processing circuitry, as will be discussed in more detail below.

Although FIG. 2C shows the platform 28 coupled to the wrist 26 of the monitored person (anyone wearing the monitoring device 20), in alternate embodiments the platform may be coupled to other areas of the user's arm. In still other embodiments, the platform may be coupled to a user's leg, such as at or near the ankle. Generally, the platform is coupled to a limb of the monitored person. Unlike many monitoring devices of the prior art, the platform of the present invention may be embodied as a single platform as opposed to multiple platforms coupled to multiple places on the user's body thereby increasing the ease of use and comfort to the user.

The monitoring device 20 may have one or more sensors mounted on the platform 28. For clarity of drawing, FIGS. 2A-2C provide illustrations of a first sensor 30 and a second sensor 32, although many sensors may be mounted to the platform 28. The sensors 30, 32 generate data based on physiological parameters of the monitored person. The first sensor 30 is of a type of sensor that must physically contact the user and is positioned within the band 22 accordingly as illustrated in the drawings. Examples of this type of sensor include piezo-electric sensors, skin conductance sensors, skin thermistors, and electromyogram (EMG) sensors. The second sensor 32 is of another type that must not contact the user, so it is positioned on top of the band away from the user. Examples of this type of sensor include ambient temperature sensors and three-axis accelerometers. Non-limiting examples of sensors and the physiological parameters based upon which they generate data include: piezo-electric sensors, which generate heart rate data; three-axis accelerometers (motion sensors) and piezo-electric sensors, which generate tremor data based on a shaking arm or leg; impedance sensors, which generate respiration data; skin conductance sensors, which generate sweat rate data; a thermistor, which generates skin temperature data; and an electromyogram (EMG) sensor, which generates muscle-electric activity data.

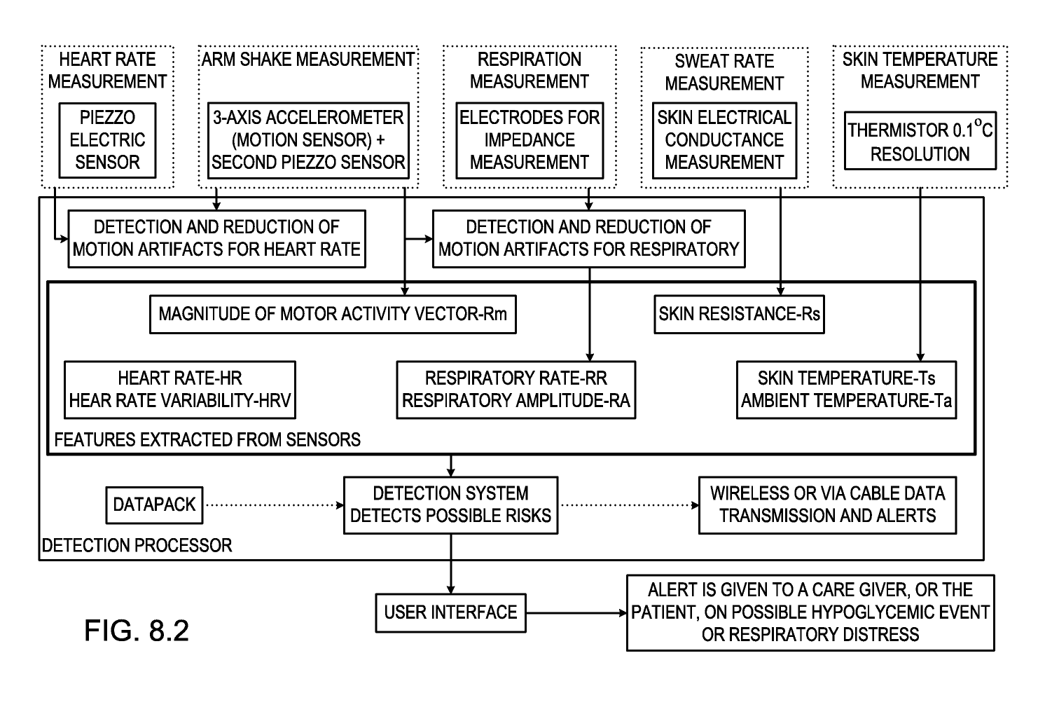

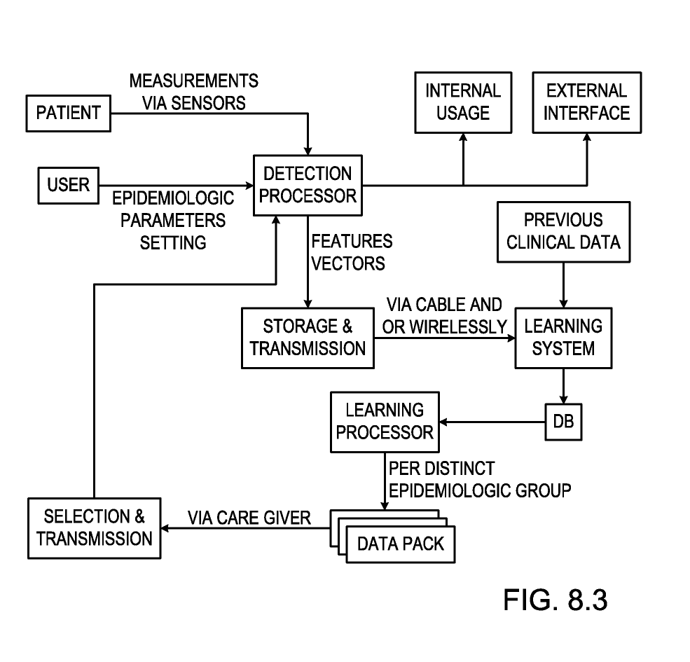

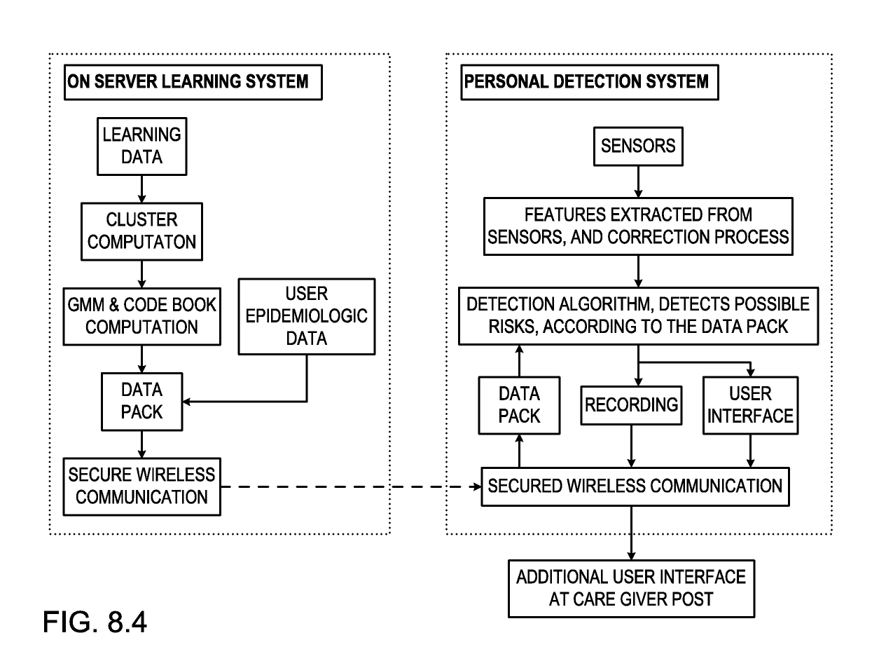

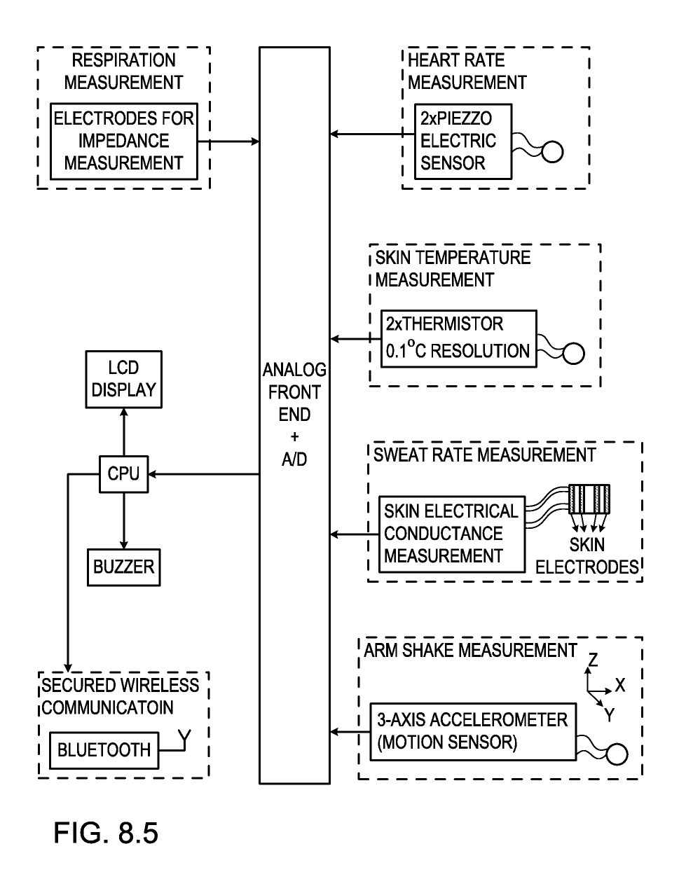

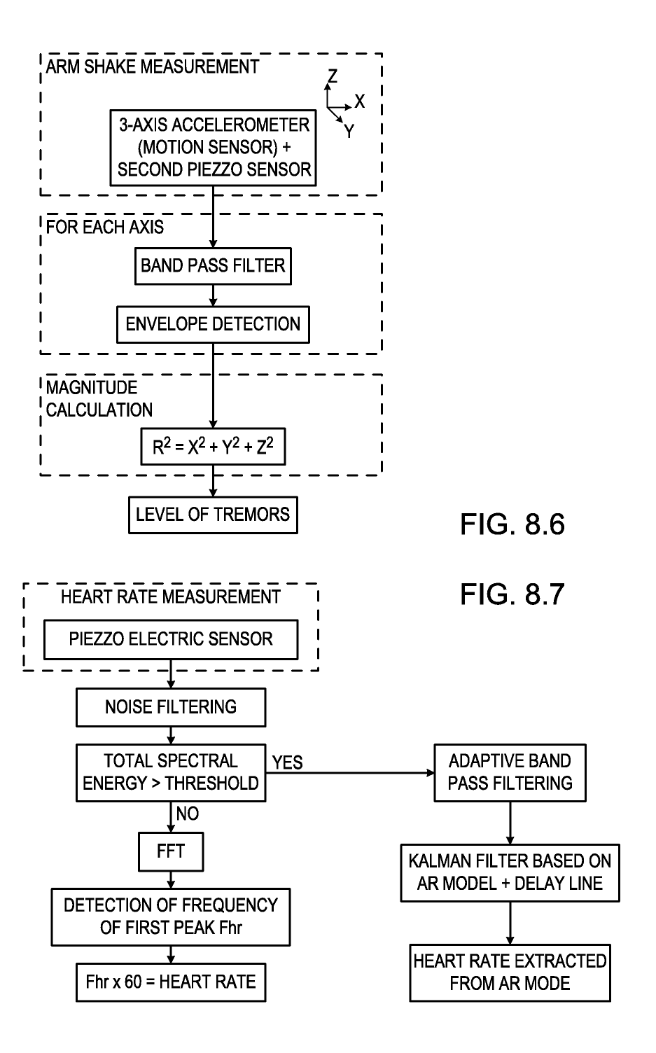

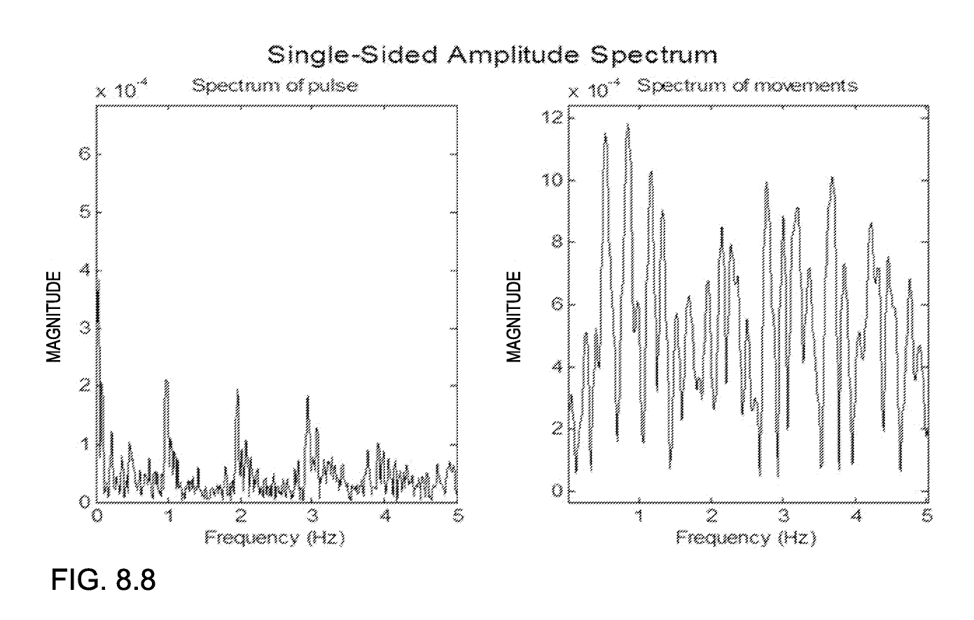

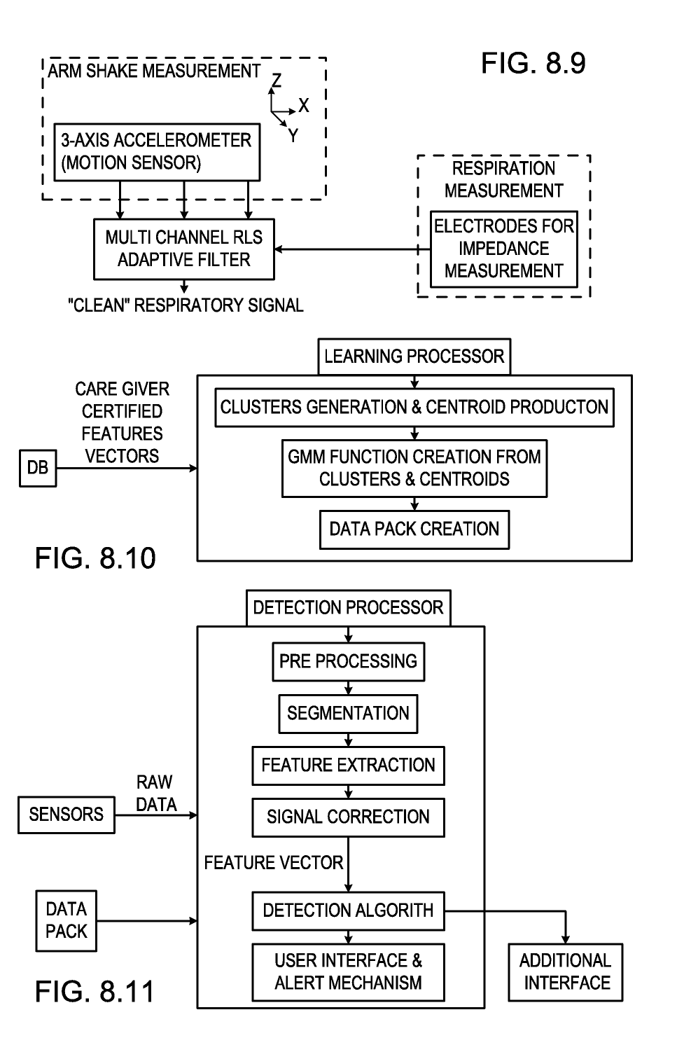

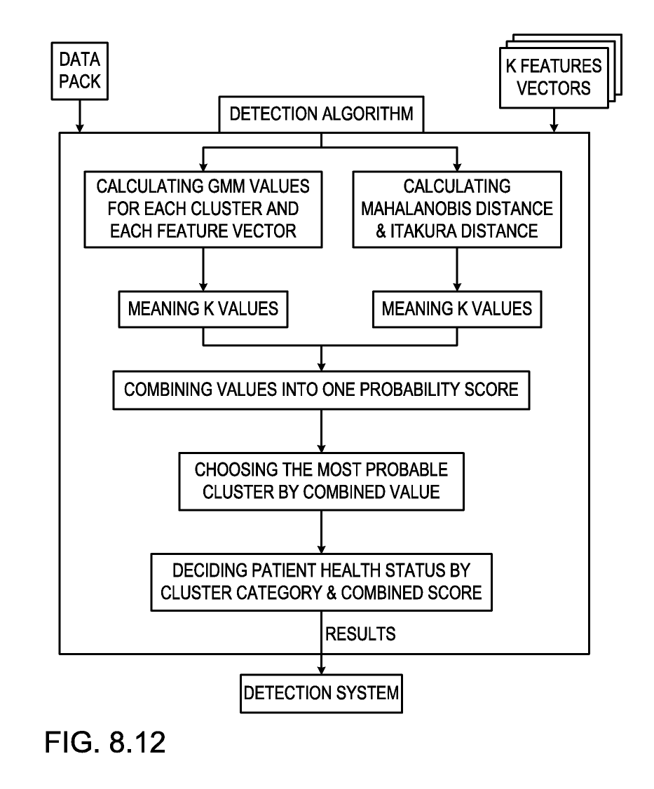

The monitoring device 20 in this embodiment has processing circuitry 34 that determines whether to activate an alarm that the monitored person's health is at risk. One non-limiting way to use sensor data to determine whether to activate an alarm is discussed below in the Appendix in the section “The Detection algorithm,” which references FIGS. 8.11 and 8.12. Alternatively, a classification algorithm may be used. As discussed in more detail below, the determination of whether to activate the alarm is based (1) on the sensor data that flows through a data path to the processing circuitry 34 and (2) on additional data. The additional data includes data that based on physiological parameters of people other than the monitored person. This data may be provided to the monitoring device 20 in the form of a data pack as discussed below. In some embodiments, the additional data may include data previously obtained from the sensors 30, 32 and stored as baseline data for future use.

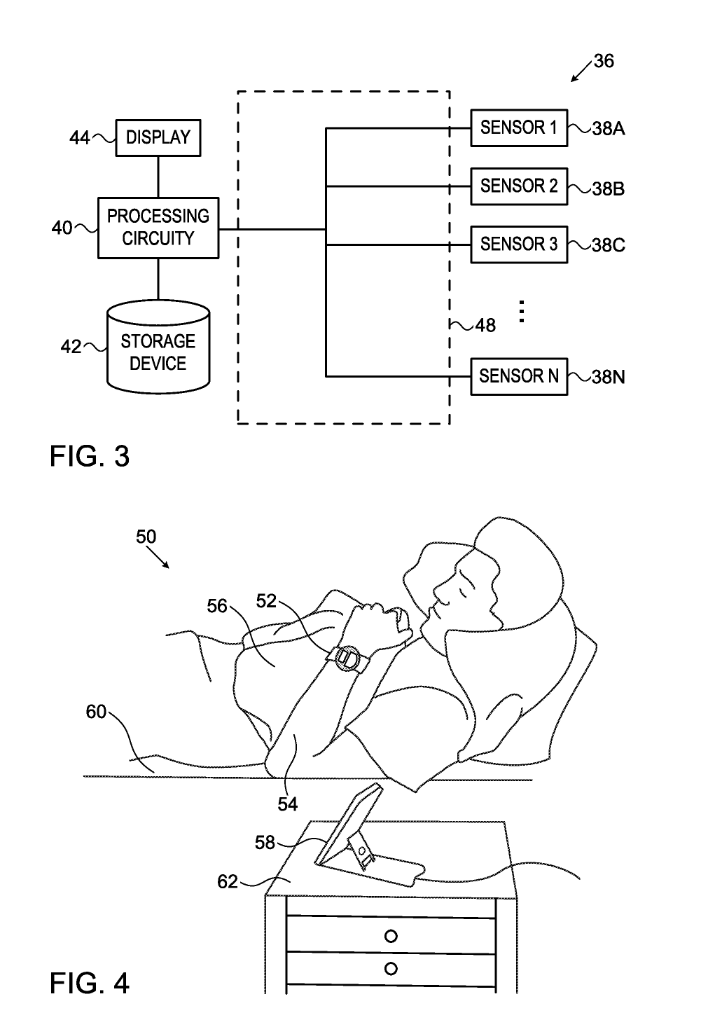

The block diagram of FIG. 3 illustrates the relationship between elements presented above. This monitoring device 36 includes sensors SENSOR 1 38A, SENSOR 2 38B, SENSOR 3 38C, . . . , SENSOR N 38N, configured to send sensor data to processing circuitry 40. The processing circuitry 40 may comprise any combination of hardware, software, and firmware using conventional or proprietary techniques or any other techniques developed to perform the functions described herein. The processing circuitry 40 may execute software instructions stored in a storage device 42. The storage device 42 may also be used in some embodiments to store as baseline data the data obtained from the sensors SENSOR 1 38A, SENSOR 2 38B, SENSOR 3 38C, . . . , SENSOR N 38N. In some embodiments, the monitoring device 36 may include a display 44 (such as display 46 mounted to the chassis 24 of the embodiment of FIGS. 2A-2C) to show information of interest, such as the status of the monitored person, the amount of charge in a battery powering the monitoring device 36, and alerts or notifications to the monitored person, as non-limiting examples. The processing circuitry 40, the storage device 42, and the display 46 may be selected from conventional technology known to those skilled in the art.

The monitoring device of FIG. 3 includes a data path 48 (shown conceptually by the broken-line box in the drawing) through which the sensor data flows from the sensors SENSOR 1 38A, SENSOR 2 38B, SENSOR 3 38C, . . . , SENSOR N 38N to the processing circuitry 40. The data path 48 may comprise electrical paths on the surface of a circuit board and/or wire leads, as non-limiting examples. In other embodiments, such some disclosed below, the data path might employ wireless technology. Generally, a “data path,” as the term is used herein, includes the elements necessary within a monitoring device to enable data from the sensors to flow to processing circuitry.

Referring back to FIGS. 2A-2C, as discussed above, the platform 28 includes both the chassis 24 and the band 22. In this embodiment, the band 22 and the sensors 30, 32 are consumables and not expected to last as long as the processing circuitry 34, which is a non-consumable. Accordingly, the chassis 24, to which the processing circuitry 34 is mounted, and the band 22, to which the sensors 30, 32 are mounted, may each be manufactured and sold separately. The band 22 may even be sold without the sensors 30, 32 but configured for the sensors to be mounted thereto. In alternate embodiments, sensors may be mounted to a chassis and processing circuitry may be mounted to a band.

As also discussed above, the sensors 30, 32 generate data based on physiological parameters of the monitored person. For clarity, the description of this embodiment presents only two sensors, but the monitoring device 20 may have more sensors, as often more types of sensor are implemented to generate data based on the various types of physiological parameters. Physiological parameters upon which the sensor data is based may include, as non-limiting examples, any combination of heart rate, heart rate variability, respiration, perspiration, skin temperature, difference between skin and ambient temperatures, motoric activity, and electrical activity in the muscles of the monitored person.

As additionally discussed above, the processing circuitry 34 of the monitoring device 20 determines whether to activate an alarm that indicates that the monitored person's health is at risk. The determination of whether to activate the alarm is based on data from the sensors of the monitoring device 20 (this data being based on physiological parameters of the monitored person) and on additional data, including data based on physiological parameters of people other than the monitored person. As discussed in more detail below, this “additional” data may be updated repeatedly, perhaps at pre-defined intervals (e.g., weekly or monthly), a usage guideline (e.g., five time during the first week and bi-monthly thereafter), or irregularly (e.g., at a caregiver's discretion). For security concerns, a caretaker may be required to provide license information and a password to effect the updating. The processing circuitry 34 may execute a classification algorithm or a detection algorithm to determine whether to activate the alarm.

The present invention may also be embodied as a non-invasive system 50 for monitoring physiological conditions, as illustrated in FIG. 4. The monitoring system 50 resembles the monitoring device 20 of FIGS. 2A-2C in that it includes a device 52 that has a platform with sensors mounted thereon and an interface. However, although the device 52 is coupled to a limb 54 of a monitored person 56, the processing circuitry 58 is not mounted to the platform of the device 52. Instead, the interface, including a wireless connectivity components which transmit and/or receive signals in accordance with protocols such as Wi-Fi or Bluetooth, allows the monitored person 56 to rest on a bed 60 or a sofa while the processing circuitry is located nearby, such as on a bedroom nightstand 62 or on a living room end table. In this embodiment, the device 52 may be manufactured and sold separately from the processing circuitry 58. The processing circuitry may include an application residing on a smartphone or a tablet, as non-limiting examples, or may be a specially-designed stand-alone unit.

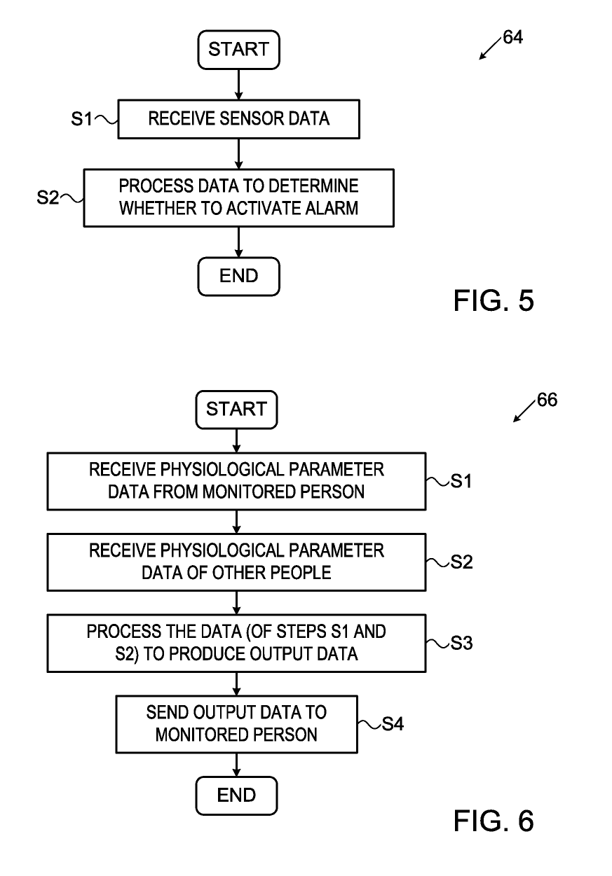

The present invention may also be embodied as a non-invasive method of monitoring physiological conditions as represented by the flow chart 64 in FIG. 5. The monitoring device 20 of FIGS. 2A-2C or the monitoring system 50 of FIG. 4 may be used in the execution of this method.

The first step is to receive data from one or more sensors (e.g., sensors 30, 32 of the monitoring device 20) mounted to a platform (e.g., the platform 24 of the monitoring device 20) that is coupled to a limb of a monitored person. (Step S1.) The sensors used in this embodiment generate data based on physiological parameters of the monitored person. As in the above embodiments, the platform may include (1) a chassis to which circuitry to process the data is mounted and (2) a band to which the one or more sensors are mounted, the chassis being mounted to the band. A display may be mounted to the chassis. Also as in the above embodiments, the physiological parameters upon which the sensor data is based include, as non-limiting examples, any combination of heart rate, heart rate variability, respiration, perspiration, skin temperature, difference between skin and ambient temperatures, motoric activity, and electrical activity in the muscles of the monitored person.

After the data are retrieved in step S1, the next step is to process the data to determine whether to activate an alarm that indicates that the monitored person's health is at risk. (Step S2.) This determination is based on the sensor data retrieved in step S1 and on additional data, which includes data that is based on physiological parameters of people other than the monitored person. As in the above embodiment, the additional data are updated repeatedly, such as at pre-defined intervals, according to a usage guideline, or irregularly. A classification algorithm or a detection algorithm may be executed in this step to determine whether to activate the alarm. In some embodiments, the additional data may include data previously obtained from the sensors and stored as baseline data for use, such as by the classification or detection algorithms.

The present invention may further be embodied as a method of assisting the monitoring of physiological conditions as represented by the flow chart 66 in FIG. 6. Reference is made briefly above to the conceptual diagram of FIG. 1 in general and to the learning module 16 in particular, which represent embodiments for which the instances of false alarms when monitoring physiological conditions of a user are reduced while nonetheless maintaining a system that is sensitive enough to detect genuine emergencies regarding the user's medical condition. A learning algorithm effectively calibrates a monitoring system to an individual user to provide this improved performance. The learning algorithm is used to generate data to effect the calibration. The data may be provided to the personal detection module 12 in the form of a data pack such as according to the embodiment disclosed next.

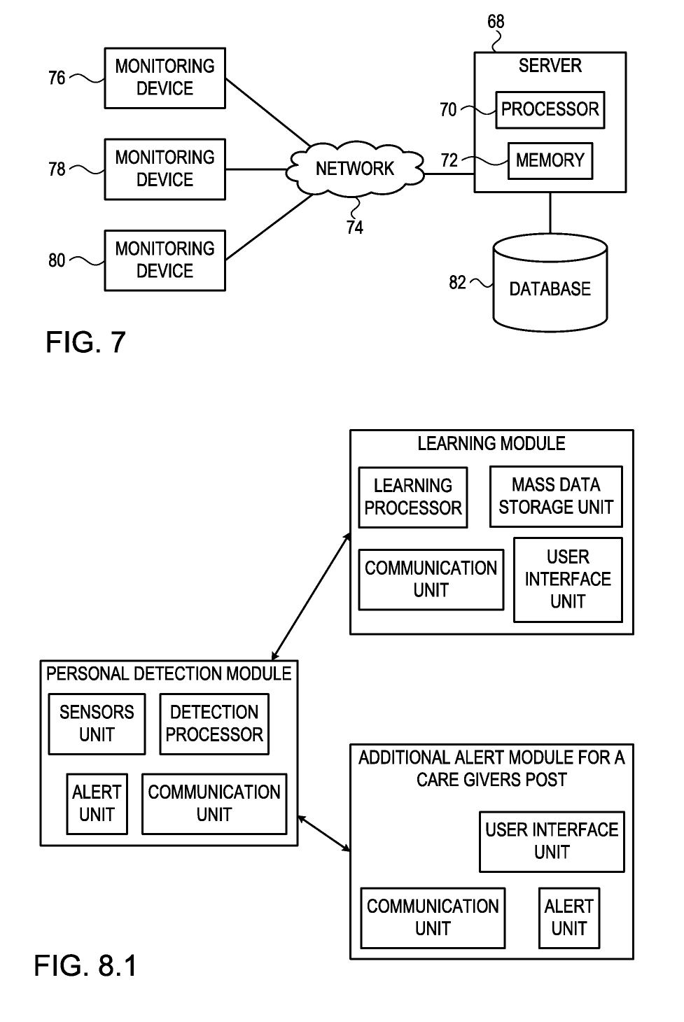

With reference again to FIG. 6, the first step of the method is to receive data based on the physiological parameters of the monitored person. (Step S1.) A non-limiting example of implementing this step is to operate the server 68 in FIG. 7 such that its processor 70 executes instructions stored in its memory 72 to receive through a network 74, such as the Internet, data being generated by one or more sensors mounted to a platform that is coupled to a limb of the monitored person. The monitored person may be using a monitoring device 76, which is constructed according to the principles discussed above with respect to FIGS. 2A-2C and 4.

The next step (which alternately may be performed before or simultaneously with step S1) is to receive data based on the physiological parameters of people other than the monitored person. (Step S2.) The other people may be using monitoring devices 78 and 80, with the monitoring devices 78 and 80 sending their data through the network 74, where the data are subsequently received by the server 68. Alternatively, the data provided to the server 68 may originate from clinical studies, in which the physiological parameters of many patients are observed to provide “group data.” In some embodiments, the server 68 only receives the group data upon user or caregiver intervention. The data may be stored in a database 82, which is operably connected to the server 68.

After the data is received in steps S1 and S2, the next step is to process the data based on the physiological parameters of the monitored person and the data based on physiological parameters of people other than the monitored person to produce output data. (Step S3.) This output data can become available as the data pack discussed briefly in discussions above. This output data may be produced using a clustering algorithm. The Appendix below presents exemplary implementations of the data pack and the clustering algorithm, such as in the section “The learning algorithm.”

After the data is processed in step S3 to produce the output data, the next step is to send the output data to processing circuitry that determines whether to activate an alarm that the monitored person's health is at risk. (Step S4.) The processing circuitry may be mounted to the platform, such as in the embodiment of FIGS. 2A-2C, or it may not mounted to the platform, such as in the embodiments of FIG. 4. The method then ends.

In alternate embodiments, in addition to receiving data based on the physiological parameters of people other than the monitored person (step S2), the method of assisting the monitoring of physiological conditions includes receiving additional data based on physiological and/or genetic parameters of the monitored person, but these additional data are not generated by the sensors mounted to the platform of the monitored person's monitoring device. As non-limiting examples, this type of data may include the height, weight, race, and/or general health condition of the monitored person, such as whether he/she has a heart condition, a respiratory condition, or diabetes. The additional data may additionally or alternatively indicate the type of diabetes or other illness, the length of time of insulin use, the type of insulin used, and/or the number of past severe events. These additional data are also processed in step S3 to produce the output data.

Some embodiments of the invention, in addition to performing the steps discussed above with reference to FIG. 6, also perform additional steps, including receiving new data based on at least one of physiological parameters of the monitored person and/or new data based on physiological parameters of people other than the monitored person. The term “new” in this context refers to data that have not yet been used and are newer. These embodiments also include the step of processing the new data to produce updated output data. According to the particular embodiment, the new data may be processed with the older data that were processed already in step S3, or they may be processed without the older data. Afterward, the updated output data are sent to the processing circuitry for use. In this fashion, monitoring devices are continually adjusted as appropriate to the individual user.

The present invention may further be embodied as a machine readable storage medium containing instructions that when executed cause processing circuitry, such as the processing circuitry 34 in FIG. 2A, the processing circuitry 40 of FIG. 3, or the processing circuitry 58 of FIG. 4, to perform the methods described above. The storage media are not illustrated in FIGS. 2A and 4 for clarity. The storage device 42 in FIG. 3 may hold the instructions as discussed above. The instructions may cause the processing circuitry to: (1) receive data from one or more sensors mounted to a platform that is coupled to a limb of a monitored person, the sensors generating data based on physiological parameters of the monitored person; and (2) process the data to determine whether to activate an alarm that the monitored person's health is at risk, the determination being based on the sensor data and on additional data including data based on physiological parameters of people other than the monitored person, the additional data being updated repeatedly.

The present invention may also be embodied as a machine readable storage medium containing instructions that when executed cause a computer, such as the server 68 in FIG. 7, to perform the methods described above. As a non-limiting example, the memory 72 may serve as the storage medium holding the instructions as discussed above. The instructions may cause the computer to: (1) receive data based on physiological parameters of a monitored person, the data being generated by one or more sensors mounted to a platform that is coupled to a limb of the monitored person; (2) receive data based on physiological parameters of people other than the monitored person; (3) process the data based on the physiological parameters of the monitored person and the data based on physiological parameters of people other than the monitored person to produce output data; and (4) send the output data to processing circuitry that determines whether to activate an alarm that the monitored person's health is at risk.

Having thus described exemplary embodiments of the invention, it will be apparent that various alterations, modifications, and improvements will readily occur to those skilled in the art. Alternations, modifications, and improvements of the disclosed invention, though not expressly described above, are nonetheless intended and implied to be within spirit and scope of the invention. Accordingly, the foregoing discussion is intended to be illustrative only; the invention is limited and defined only by the following claims and equivalents thereto.

### APPENDIX

The following reproduces the content of the U.S. provisional patent application upon which the present application claims priority:

PROVISIONAL PATENT APPLICATION FOR An improved system for detection and alert physiological risks during sleep

### ABSTRACT

A non-invasive system for detecting and alerting of several potentially dangerous physiological risks during sleep time. Some examples of these physiological risks may include nocturnal hypoglycemia, sleep time respiratory distress such as sleep apnea or asthmatic seizure, nocturnal epileptic seizures, Arrhythmias, sudden death syndromes, pulmonary edema, among others. The system will have the ability to record and transmit the sensor's readings to additional devices such as tablets and care giver data systems.

The device may include sensors for monitoring physiological parameters regarding skin temperature, perspiration, motor activity, respiratory rate and heart rate and blood pulsation with correction for motion artifacts, all can be monitored from the wrist or a limb or any comfortable location on the body. The device may include a detection processor performing a detection algorithm which may determine if the reading's suggests a physiological risk and if so may alert the user and act according to the programmed scenarios. The device may also include a communication unit that may have the ability to transmit the recordings via cable or wireless communication, on predefined scenarios or user intervention. The system may also include a learning processor having the ability to update the detection algorithm according to a learning algorithm, capable of learning the characteristics of each type of physiological event from prior medical knowledge regarding physiological measurements and epidemiologic parameters.

### FIELD OF INVENTION

The present invention generally relates to the field of physiological measurement. More particularly, the present invention relates to a system and method for monitoring physiological parameters associated with physiological risks of an individual during sleep time (see Abstract).

### DETAILED DESCRIPTION OF THE PRESENT INVENTION

In accordance with the present invention an improved method and system for detection and alerting of potentially dangerous physiological events during sleep is provided. The system of the present invention may provide for reducing the rate of false alarms, which might cause unnecessary disturbance of the patient's sleep, and yet retaining a high sensitivity and reliable detection of possible health risks.

### Introduction

The system of the present invention may include three different hardware modules: 1. A personal detection module, which may include a detection processor, 2. Additional alert module for care givers, which may include a user interface and means for audio and displayed Alerts 3. A server-based learning module which may include a learning processor.

An example for the hardware modules and internal units for each module, according to an embodiment of the present invention, is displayed in FIG. 8.1 (FIG. 1 in the provisional application, which illustrates hardware modules of an embodiment of the present invention).

All the modules of the system may include a communication unit allowing data transfers between the modules.

The method of the present invention may include 3 main functions: 1. physiological parameters measurement and artifacts correction 2. physiological risk detection 3. external learning.

### Function Introduction

The first function may measure and remove motion artifacts from the following: i. heart rate-HR and heart rate variability-HRV; ii. motoric activity level-Rm; iii. skin temperature Ts compared to ambient temperature Ta; iv. Sweat level according to skin resistance-Rs; v. respiratory rate RR and amplitude Ra.

The Second function is the detection algorithm which may use a data pack and a set of similarity criterions for the detection process. The data pack may include, but not limited, to a set of probabilistic functions such as GMM (Gaussian mixture model) functions, and a code book which may include a set of centroids representing clusters data. The data pack represents references for possible health risks. The probabilistic functions set and the code book may be created externally or internally by the learning algorithm.

The first two functions may be performed by the detection module. A functional diagram of an exemplary detection system, according to an embodiment of the present invention, is displayed in FIG. 8.2 (FIG. 2 in the provisional application, which illustrates a functional diagram of an exemplary personal detection system).

The third function is a learning function, and may include a clustering algorithm such as vector quantization for codebook generation, and additional algorithms for creating probabilistic functions, for example GMM functions, according to prior measurements of the system's parameters or prior physiological knowledge. The algorithm's output may include, but not limited to, a set of probabilistic functions, such as GMM functions, and a set of centroids for the detection algorithm. This function may be performed by the learning processor.

The flow chart of an exemplary learning function, according to an embodiment of the present invention, is displayed in FIG. 8.3 (FIG. 3 in the provisional application, which illustrates a Functional diagram of an exemplary personal detection system.)

The user, be it a care giver or the patient itself, may set the epidemiologic parameters which may be transmitted along with the patient's measurements to the detection processor. The detection processor may use the data for internal use (determining the patient medical situation) and may also transfer the data to the learning processor via the storage and transmission units. The learning processor is able to store all readings and can create a data pack which may be used for the calibration of the device per epidemiologic group. The care giver may transmit the settings from the learning processor into the detection processor for detection usage.

### General Scheme

FIG. 8.4 (FIG. 4 in the provisional application, which illustrates a working principle of the system) displays a general scheme for an exemplary implementation of a system according to an embodiment of the present invention.

### The Hardware

### The Personal Detection Module

The personal detection module, which may be included in a wrist-watch device, or alternatively, as a module which may be attached to the human body on other anatomical locations. This is a measuring and computing module, equipped with sensors which are connected to respective electrical circuits. The detection module may be structured in such a manner that it may provide for the measurement of multiple parameters from a single anatomical location.

The sensors may include:
1. A number of electrodes, for example 4 electrodes, which may be used for sensing physiological parameters such as sweat level, respiratory depth and rate and blood pulsation.
2. Thermistors which are placed in contact with the skin, and on the chassis
3. Piezo-electric pressure sensors, in contact with the skin, and attached to the sensors which are placed on the skin as a second layer.
4. A three-axis accelerometer, placed on the chassis near the skin.

The unit may also include an A/D component which is able to digitize the data from the sensors, and may be further connected to a detection processor which may have a programmable memory, and a digital communication interface able to connect to external devices by means of a cable or wireless communication. The detection processor may provide for performing operations such as features extraction from the sensors data, for example: calculation of the heart rate and reduction of motion artifacts. The detection processor may also provide for performing operations required for implementing the algorithm for detection of health risks, and activating the buzzer and alarm accordingly. The detection processor is installed on an electrical circuit inside the module. The power source of this unit may be implemented by rechargeable batteries.

FIG. 8.5 (FIG. 5 in the provisional application, which presents a block diagram of the measuring unit) displays an exemplary technical block diagram of the components of the personal detection system, according to an embodiment of the present invention.

### The Sensor Array

The system may monitor skin temperature using two sets of thermistors. One is attached to the skin for measuring skin temperature, and the other may be placed on the detection module for measurement of ambient temperature. External heat or cold might impact skin temperature, therefore the detection processor may add a weight factor to the skin temperature measurement, Ts, according to the measured ambient temperature.

The system may also monitor sweat level by measuring skin resistance. Skin resistance may be measured by applying a small DC electric current to the skin and measuring the resulting voltage on a set of electrodes. Relative high ambient temperature may cause high level of sweat with no regards to physiological risks. Therefore the detection processor may also add a weight factor according to ambient temperature, in order to reduce the chance of false alarm for a sleep time health risk.

The system may also monitor heart rate (HR) and heart rate variability (HRV) and may use a 2-layer piezo-electric sensors array. The sensors may be structured in 2 layers, each layer consist of Printed Circuit Board (PCB) with a piezo-electric pressure sensor. The upper layer, may be attached to the skin of the human body, in order to measure the heart pulse, and the unavoidable motion artifacts, this is termed the “primary HR sensor”. The lower layer may be placed under the first layer in order to measure only the motion artifacts without measuring the heart pulse, and is termed the “reference HR sensor”.

Motoric activity may be measured by several types of sensors, two possible examples are given here. The first type of sensor may be a three-axis accelerometer that may be used to measure the movements of the human body. This includes large motion, seizure like motion, and also tremors. The second sensor may be a piezo-electric pressure sensor, which is the “reference HR sensor”, and is highly sensitive to smaller tremors.

The system may also monitor respiration rate and respiration depth (also known as respiration amplitude). Respiration signal may be measured by means such as Bio-Impedance method, wherein, for example, a small ac current may be injected to the skin by electrodes, and the resulting voltage may be measured in order to measure the electrical impedance of the body volume between the measuring electrodes.

### The User Interface for the Personal Detection Module

A user interface may be further implemented in the detection module which may provide personal alerts for the patient being monitored. The alerts may be performed by utilizing a LCD and a buzzer. It is possible that the patient may be awakened from his sleep as a result of an alert given by the system, but he is unable to take action needed in this situation. Therefore, the user interface may further include a distress button to allow a patient to call for assistance.

### The Additional Interface for Caregivers

The Additional interface module, which may be capable of communication with the detection module, may be based on an embedded platform such as a personal computer (PC), or a tablet computer. The module may also be further implemented on a specially designed device. This device may be structured as a case with a large display, keyboard and audio alerts, and may be capable of wireless communication with the detection module. The module may further include a Graphical User Interface (GUI) and may allow configuration of the device working parameters, real time monitoring of the patient by a care providing person, and off line analysis of the patient's physiological information. For example, parents may monitor the symptoms of a sleeping diabetic child, and check him for a possible hypoglycemic event, or check the breath of an asthmatic child, from another room without waking up the child. Off line analysis of the patient's symptoms may allow professional medical personal to adjust treatment, for example it may allow doctors to give a more personalized medication regime.

### The Learning Module—Server Side

The server side of the system may include the computer, which is the learning processor, it may perform the learning algorithm and creates the data pack needed to update the detection module.

### The Main Functions

### The Feature Extraction and Signal Correction

The instantaneous level of skin temperature and ambient temperatures may be repeatedly measured at a predefined repetition rate, termed as “temperature sample rate”. The representing value may be the averaged skin temperature and the averaged ambient temperature of the current sample with previous samples. The number of samples for averaging may be predefined and software configurable by the user interface. A difference in averaged skin temperature in comparison to ambient temperature over a predefined and software configurable time duration, may be considered as parameter for the system.

Sweat level is extracted from the instantaneous level of galvanic skin resistance (GSR) which may be repeatedly measured at a predefined repetition rate, termed as “GSR sample rate”. Skin resistance may be repeatedly measured at these given time points, and the representing value may be the averaged skin resistance of recent previous samples or any other combination of the previous samples. The number of samples for averaging may be predefined and software configurable by the user interface. The level of skin resistance may be scaled according to the current level of ambient temperature. When the ambient temperature is lower than a predefined temperature, it is unlikely that such perspiring is caused by the ambient temperature. When the ambient temperature is higher than a predefine threshold, the weight of skin resistance on the detection of sleep time health risk event may be reduced. This weight factor may be a part of the detection system. The threshold for this event may be software configurable by the user interface. The instantaneous level of skin resistance may be compared to a basal scale, and the change from the basal scale is of interest in the disclosed system. This scale may be set according to know typical basal levels of GSR, or alternatively can be manually set by the user interface in order to adjust to a specific patient.

Motor activity may be extracted from the signals of the three axes accelerometer, which may be repeatedly sampled along each axis at a predefined sample rate, termed as “Motor activity sample rate”. Tremors and seizures are rhythmic repeating motions, and therefore can be characterized as signals in a specific frequency range. The system may apply a band pass filter, which allows only the usage of signals in a predefined frequency range, this frequency range may be software configurable by the user interface. The level of activity along each axis may be represented by the amplitude of these signals. The amplitude may be extracted by an envelope detector, which may be implemented in software. The instantaneous level of motor activity may be the magnitude of the 3-axis vector of activity level, and may be calculated according to the following exemplary formula: R²=X²+Y²+Z². A flow chart for an exemplary calculation process for the instantaneous level of motor activity, according to an embodiment of the present invention, is displayed in FIG. 8.6 (FIG. 6 in the provisional application, which provides a Flow chart example for calculation of the level of tremors).

The representing value may be the averaged motor activity of recent samples or any combination of the samples. The number of samples may be predefined and software configurable by the user interface.

The heart rate (HR) and heart rate variability (HRV) may be extracted from the signals of the 2-layers piezo electric sensors array, which may be repeatedly sampled at a predefined sample rate, termed as “Heart Pulse sample rate”. A flow chart of an exemplary algorithm of heart rate calculation, according to an embodiment of the present invention, is displayed in FIG. 8.7 (FIG. 7 in the provisional application, which provides a flow chart for the algorithm for heart rate measurement).

The algorithm may detect and reduce motion related artifacts in the measurement of heart pulsation. The algorithm may include a MDU (Motion Detection Unit) and an ARU (Artifact Reduction Unit) used for signal segments contaminated with motion artifacts, and also a simpler calculation for artifact free signal segments.

The MDU may detect motion artifacts according to the distribution of spectral energy. An example for a typical spectral energy distribution of an artifact clean signal, and a motion contaminated signal, according to an embodiment of the present invention, is given in FIG. 8 (FIG. 8.8 in the provisional application, which provides an example for spectral energy distribution in the frequency domain between, on the left, an artifact clean signal, and, on the right, a motion contaminated signal.

A clean signal may be characterized by a typical harmonic structure, starting with the first frequency component which is in the frequency of the heart rate, and followed by harmonic components at frequencies which are multiplicands of the heart rate. Motion artifact will add frequency components which are not typical to the structure of heart pulsation signal, and will therefore result in a large increase of total spectral energy (TSE). The heart rate signal from the primary sensor may be segmented into time windows, the duration for each window may be predefined and programmable by the user interface. For each time window the TSE may be calculated, and compared to a threshold. If the TSE is higher or equal than the threshold, this segment of the signal may be considered a motion contaminated signal, otherwise it may be considered clean of artifacts. This may be performed by the MDU, if the MDU detects motion artifacts than the heart rate calculation may be performed by the ARU. If the signal is considered clean, than the heart rate calculation may be performed by finding the first non-DC peak of the frequency spectrum termed Fₕᵣ, which may be calculated by means of FFT (Fast Fourier Transform), and the heart rate is:

HR=Fₕᵣ·60

The ARU may apply a two stages algorithm for motion artifacts reduction. The first stage may apply multiple band pass filters (BPF), based on the harmonic structure of the heart pulsation signal. Each BPF may be centered at a known harmonic frequency of the heart pulsation signal according to the last measured “clean” signal segment. This may be done under the assumption that the change in heart rate is very small between up to a predefined number of adjacent segments. The width of each BPF allows detection of a small change in the heart rate from the last known clean segment. This filtering method may be performed on both the primary sensors, and the reference sensors. The second stage of the ARU algorithm may be adaptive filtering, by means of an adaptive kalman filter (AKF) for example, which may consist of two sets of inputs, the primary pulse signal, which may include the heart pulsation signal and motion artifacts, and a reference signal with motion artifacts only. The adaptive filter may be a model based filter, and therefore may apply a mathematical model representing the pulsation signal, for example an AR (Auto-Regressive) model, for estimating the heart rate pulsation signal, and also a noise model for the additive motion artifact which may be based on the reference signal. In this method, only the clean heart rate may be estimated according to the pulse signal model, and the motion artifacts may be ignored. This filter may further apply delay lines on the reference signals to adjust for time delays between the primary sensors signals and the reference signals. The delay line may be implemented by a filter with a constant gain of 1, and an adaptive phase response. This may be adaptively controlled by the ARU algorithm according to the lag time of maximum correlation in the cross correlation function between the primary signal and the reference signal for each signal segment.

The Respiratory signal may be sampled at a predefined sample rate, termed as “Respiratory sample rate”. The Respiratory signal may be segmented into time windows, the duration for each window may be predefined and programmable by the user interface. Each signal segment may be fitted into a representing model such as an AR model using the Levinson Durbin algorithm or other suitable algorithm known in the art. The respiratory rate (RR) and respiratory amplitude (RA) may be estimated by the coefficients and parameters of the model.

Respiratory signals might also be contaminated with motion artifacts, and therefore a Respiratory Motion Artifact Reduction algorithm may be further implemented in an embodiment of the present invention. Motion artifacts may be reduced by a multi channel RLS (Recursive Least Square) adaptive filter, or using other types of digital filters known in the art and which may provide adaptive filtering. This method may reduce artifacts which may be separately measured by the three-axis accelerometer (which may be used for motion measurement) or any other sensor which is able to measure motion. FIG. 8.9 (FIG. 9 in the provisional application, which illustrates a respiratory motion artifact reduction method) displays a block diagram of an exemplary motion artifacts reduction method for respiratory signals, according to an embodiment of the present invention.

### The Learning Algorithm

The learning algorithm may use clustering methods such as vector quantization and guided learning, and may apply different methods known in the art to determine similarity such as Euclidean distance and Itakura distortion measure. The learning algorithm may be performed by the learning processor according to exemplary flow chart displayed in FIG. 8.10 (FIG. 10, the provisional application, which provides a flow chart for the learning algorithm) according to an embodiment of the present invention.

A combination of a signal's values of each feature with a single value of each epidemiological feature is considered a single training point in the training data. Let us define X={x₁, . . . , xₙ} as the set of points each point is the measured physiological values, the patient epidemiologic values & the physiological condition (healthy or the type of the physiological risk), and let us define C={c₁, . . . , cₖ} as the clusters outputted by the learning algorithm with which we create the set of probabilistic functions, F=(ƒ₁, . . . , ƒₖ) utilizing methods such as the expectation maximization method. The created probabilistic functions and the distance measures may be used as an input a point without the physiological type value. Note that each of the centroids or clusters has a physiological type for it represents a homogenic group.

The clusters sets are dividing the learning data according to the clustering algorithm's criterions in order to reach to optimal division of the feature space required for the detection algorithm to detect each of the physiological risks.

Finally the algorithm divide's the clusters and probabilistic functions, to classes denoting the patient physical health status and risk type. The collection F and the set of centroids of the clusters with the division to classes are packed as the data pack, which may be sent to the detection processor.

### The Detection Algorithm

The user physical health status may be determined using a detection algorithm which may utilize the data pack from the learning algorithm and pre-defined user epidemiologic data. A combination of a signal's values of each feature with a single value of each epidemiological feature is considered a single point at time t and will be marked as xₜ. An example for the flow chart of the detection algorithm, according to an embodiment of the present invention, is given in FIG. 8.11 (FIG. 11 in the provisional application, which provides a flow chart of an example for the algorithm performed by the detection processor).

The detection processor may perform noise filtering using band pass filters as a pre processing stage on the raw data from the sensors. This may be followed by segmentation into time frames which may be required for extraction of features such as heart rate and respiratory parameters. Signal correction for artifact reduction may also be performed on the extracted features. FIG. 8.12 (FIG. 12 in the provisional application, which provides a flow chart of an example for the algorithm performed by the detection processor) displays an example for the process in which the given features are fed into the detection algorithm, according to an embodiment of the present invention.

The detection algorithm calculates the similarity measure, such as the Mahalanobis distance, from the point xₜ to each of the cluster's centroid cⱼ, the distance is marked [d(c]ij,xᵢt). Alternatively the algorithm will evaluate the value of each probabilistic function at the given point ƒⱼ(xₜ). After which an average distance may be calculated for a set of k consecutive points or any other calculation involving a set of k points.

The detection algorithm may select the cluster index j for which the value of the averaged value of probabilistic functions for k previous points

$$p = \frac{[ {\sum }_{i = 1}^{k} {f}_{j} ( {x}_{i} ) ]}{k}$$

is maximized compared to the other averaged values of the probabilistic functions for k previous points. Alternatively the detection algorithm may select the cluster index j for which the value of the averaged distance—

$$\frac{[ {\sum }_{i = 1}^{k} d ( {x}_{i} , {c}_{j} ) ]}{k}$$

is minimized compared to the other centroids.

The index j may represent a cluster which is assigned to a class defined by a health status. This is the detected physiological condition of the patient.

1. A non-invasive device for monitoring physiological conditions, the device comprising:
a platform configured for coupling to a limb of a monitored person;
one or more non-invasive sensors mounted on the platform, the sensors operative to generate data based on physiological parameters of the monitored person; and
a data path through which the sensor data flows to processing circuitry that determines whether to activate an alarm that the monitored person's health is at risk;
wherein the determination of the processing circuitry is based on the sensor data and on additional data including data based on physiological parameters of people other than the monitored person, the additional data being updated repeatedly;
and wherein the sensor data is provided only by the one or more non-invasive sensors mounted on the platform.

2. (canceled)

3. The device of claim 1, wherein the platform includes (1) a chassis to which the processing circuitry is mounted and (2) a band to which the one or more sensors are mounted, the chassis being mounted to the band.

4. (canceled)

5. The device of claim 1, wherein the physiological parameters upon which the sensor data is based include at least two of heart rate, heart rate variability, respiration, perspiration, skin temperature, difference between skin and ambient temperatures, motoric activity, and electrical activity in muscles of the monitored person.

6-11. (canceled)

12. The device of claim 1, wherein the processing circuitry executes a classification algorithm to determine whether to activate the alarm.

13. The device of claim 1, wherein the processing circuitry executes a detection algorithm to determine whether to activate the alarm.

14. The device of claim 1, wherein the processing circuitry is mounted to the platform.

15. A non-invasive system for monitoring physiological conditions, the system comprising:
the device of claim 1;
wherein the processing circuitry is not mounted to the platform.

16. The system of claim 15, wherein the physiological parameters upon which the sensor data is based include at least two of heart rate, heart rate variability, respiration, perspiration, skin temperature, difference between skin and ambient temperatures, motoric activity, electrical activity in muscles of the monitored person.

17-22. (canceled)

23. The system of claim 15, wherein the processing circuitry executes a classification algorithm to determine whether to activate the alarm.

24. The system of claim 15, wherein the processing circuitry executes a detection algorithm to determine whether to activate the alarm.

25. A non-invasive method of monitoring physiological conditions, the method comprising:
receiving data from one or more non-invasive sensors mounted to a platform that is coupled to a limb of a monitored person, the sensors generating data based on physiological parameters of the monitored person; and
processing the data to determine whether to activate an alarm that the monitored person's health is at risk, the determination being based on the sensor data and on additional data including data based on physiological parameters of people other than the monitored person, the additional data being updated repeatedly;
wherein the sensor data is provided only by the one or more non-invasive sensors mounted on the platform.

26. The method of claim 25, wherein the platform includes (1) a chassis to which circuitry to process the data is mounted and (2) a band to which the one or more sensors are mounted, the chassis being mounted to the band.

27. (canceled)

28. The method of claim 25, wherein the physiological parameters upon which the sensor data is based include at least two of heart rate, heart rate variability, respiration, perspiration, skin temperature, difference between skin and ambient temperatures, motoric activity, and electrical activity in muscles of the monitored person.

29-34. (canceled)

35. The method of claim 25, wherein
the processing of the data includes executing a classification algorithm to determine whether to activate the alarm.

36. The method of claim 25, wherein the processing of the data includes executing a detection algorithm to determine whether to activate the alarm.

37. A method of assisting the monitoring of physiological conditions, the method comprising:
receiving data based on physiological parameters of a monitored person, the data being generated only by one or more non-invasive sensors mounted to a single platform that is coupled to a limb of the monitored person;
receiving data based on physiological parameters of people other than the monitored person;
processing the data based on the physiological parameters of the monitored person and the data based on physiological parameters of people other than the monitored person to produce output data; and
sending the output data to processing circuitry that determines whether to activate an alarm that the monitored person's health is at risk.

38. The method of claim 37, wherein the platform includes (1) a chassis to which circuitry is mounted to process the data based on the physiological parameters of the monitored person and (2) a band to which the one or more sensors are mounted, the chassis being mounted to the band.

39. (canceled)

40. The method of claim 37 further comprising:
receiving additional data based on physiological and/or genetic parameters of the monitored person, said additional data not being generated by the sensors mounted to the platform;
wherein said additional data is also processed to produce the output data.

41. The method of claim 37 further comprising:
receiving new data based on at least one of physiological parameters of the monitored person and/or new data based on physiological parameters of people other than the monitored person;
processing the new data to produce updated output data; and
sending the updated output data to the processing circuitry.

42. The method of claim 40 further comprising:
receiving new data based on at least one of physiological parameters of the monitored person, additional new data based on physiological and/or genetic parameters of the monitored person and not being generated by the sensors mounted to the platform, and/or data based on physiological parameters of people other than the monitored person;
processing the new data to produce updated output data; and
sending the updated output data to the processing circuitry.

43-48. (canceled)
